(19) United States
(12) Patent Application Publication
(10) Pub. No.: US 2024/0285633 A1
(43) Pub. Date: Aug. 29, 2024

(54) PROLONGED RELEASE TOFACITINIB COMPOSITIONS

(71) Applicant: Synthon B.V., Nijmegen (NL)

(72) Inventors: Manuel GAGO GUILLAN, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 18/566,329

(22) PCT Filed: Jun. 3, 2022
(86) PCT No.: PCT/EP2022/065209

§ 371 Date: Dec. 1, 2023

(30) Foreign Application Priority Data
Jun. 4, 2021 (EP) 21177809.7

(51) Int. Cl. A61K 31/519; A61K 9/20; A61K 9/28

(52) U.S. Cl. CPC A61K 31/519; A61K 9/2009; A61K 9/2013; A61K 9/2018; A61K 9/2054; A61K 9/2866

(57) ABSTRACT

The present invention relates to a monolithic tablet composition for oral administration of tofacitinib, or a pharmaceutically acceptable salt thereof.

### BACKGROUND OF THE PRESENT INVENTION

Tofacitinib or (3R,4R)-4-methyl-3-(methyl-7H-pyrrolo [2,3-d]pyrimidin-4-ylamino)-ß-oxo-1-piperidinepropanenitrile, citrate salt (1:1), of the formula:

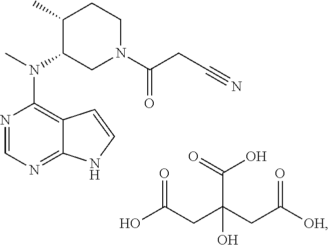 [SMILES: C[C@@H]1CCN(C(=O)CC#N)C[C@@H]1N(C)c1ncnc2[nH]ccc12 ; O=C(O)CC(O)(CC(=O)O)C(=O)O]

is a reversible inhibitor of the Janus kinase family of kinases (JAK1, JAK2, JAK3 and Tyrosine Kinase 2 (TyK2)). Tofacitinib has been disclosed in WO2001042246.

Tofacitinib is indicated for the treatment of adult patients with moderately to severely active rheumatoid arthritis who have had an inadequate response or intolerance to methotrexate. It is marketed as an extended release tablet under the brand name XELJANZ XR® (Pfizer Products Inc.). The tablets are based on osmotic pump technology, wherein the osmotic pressure is used to deliver the tofacitinib at controlled rate. The tablet insert for XELJANZ XR® tablet, describes the tablet as “a pink, oval, extended release film-coated tablet with a drilled hole at one end of the tablet band”.

XELJANZ XR® tablet is a controlled-release formulation, which provides more favourable pharmacokinetic profiles (e.g. reducing the peak variation of drug concentration levels), so reducing the side effects and achieving better patient compliance.

XELJANZ XR® drug release profile is very complicated combining different order kinetics. XELJANZ XR® formulation is described in WO2014147526; the formulation is an osmotic pump consisting of a coating made of an insoluble polymer, cellulose acetate, and a core containing tofacitinib citrate, sorbitol, hydroxyethyl cellulose, co-povidone and magnesium stearate. This coating is such that tofacitinib is substantially entirely delivered through the delivery hole, in contrast to delivery via permeation through the coating. The solute concentration gradient, which provides the osmotic force driving the delivery of the drug through the drilled hole, can be maintained constant when solute saturation is present in the tablet core. As the tablet content comes out, solute concentration declines and as well the gradient and the osmotic force driving the drug release.

The typical orifice size in osmotic pumps ranges from about 600 μm to 1 mm. A nominal 600 μm hole usually has a ±100 μm tolerance on diameter, and an allowable ellipticity of 1.0 to 1.5. Although holes of these characteristics and tolerances can be obtained by mechanical means, there is no mechanical method able to work at high manufacturing rates consistent with pharmaceutical manufacturing processes.

In contrast, laser tablet drilling can lead to throughput rates of up to 100,000 tablets/hour having the necessary dimensional tolerances and cosmetic appearance. As a result, laser drilling has become the technology of choice for this type of orifice production.

This technology also requires accepted-rejected system in order to check if the drilled hole on the surface of the tablet meets the specifications. The reject mode is activated as soon as a failed tablet is sensed by the vision system, which causes one or two tablets ahead of the rejected unit to be expelled as well. The reject state only switches off when the system verifies that five tablets in a row meet pass criterion. An additional presence sensor downstream from the blow off verifies that no tablets are passing through the system when the reject condition is set to “on”.

Therefore, the required technology for the manufacturing of the osmotic pump delivery systems is significantly expensive, which is a disadvantage and an economic barrier for many companies.

WO 2012/100949 provides an oral dosage form for modified release comprising tofacitinib and a non-erodible material. In this patent application a monolithic tablet containing a non-erodible material and other components such as pore formers is claimed. The main disadvantage of this type of delivery systems is the difficulties of the water to penetrate through the material, leading to slow hydration rates. This may lead as result the incomplete dissolution of the drug substance if the centre of the tablet core remains unwetted.

WO 2014/174073A1 discloses a sustained release formulation for oral administration comprising tofacitinib, a hydrophilic polymer and an alkalizing agent. The alkalizing agent is proposed for reducing API solubility in acidic pHs obtaining a non-pH dependent release formulation. Alkalizing the tablet core aims to reduce the release of the active ingredient at low pHs where it is more soluble; however, the decrease of the active ingredient solubility by alkalizing the tablet core can limit the drug release at high pHs (for instances at the small intestine) impacting on the bioavailability of the drug substance.

WO 2021/038014A1 discloses a controlled release composition for oral administration comprising tofacitinib and a coating comprising a water-insoluble polymer and a pore former in a specific ratio.

There is still need of finding an additional oral formulation of tofacitinib which overcome the problems of the prior art, is advantageously manufactured and is bioequivalent to the commercial tofacitinib tablet XELJANZ XR®.

### DETAILED DESCRIPTION OF THE PRESENT INVENTION

The present invention relates to a monolithic tablet that is advantageously manufactured and is able to provide a similar dissolution release rate of tofacitinib than the commercial tablets having an osmotic pump.

As used herein the term “monolithic tablet” refers to a tablet comprising a swellable hydrophilic matrix that delivers the drug in a controlled manner over a long period of time.

A first aspect of the invention relates to a controlled release pharmaceutical tablet comprising:
a) A core comprising tofacitinib or a pharmaceutically acceptable salt thereof and a water soluble pH independent gelling control release polymer;
b) A coating in an amount of 2.5% to 35.0% w/w in relation to the core tablet weight comprising a water soluble pH independent gelling control release polymer;
and the pH independent gelling control release polymer in the core and in the coating has a viscosity grade in a range from 50 to 150 cP in 2% solution in water at 20° C.

The dissolution profile provided by the osmotic pump of tofacitinib marketed tablet initially it exhibits a short lag time where no drug release takes place. This short lag time corresponds with the diffusion of water through the semi-permeable membrane and the hydration of the tablet core. Afterwards, zero-order kinetic release occurs due to the sustained solute concentration gradient between the tablet core and the dissolution medium. The solute concentration gradient, which provides the osmotic force driving the delivery of the drug through the drilled hole, can be maintained constant whereas solute saturation takes place in the tablet core. As the tablet content come out, the solute concentration declines and so the gradient and the osmotic force driving drug release. Ultimately, as a consequence of the decrease of the solute concentration in the tablet core, the dissolution profile shows first-order kinetic release after 3 hours.

Hydrophilic matrix technology has been widely used for oral controlled delivery of various drugs. As well the combination of barrier membrane and hydrophilic matrix system has been utilized as a strategy to modulate drug release from hydrophilic matrices and to reduce the overall variability in release. However, it is difficult particularly for very soluble compounds to apply this technology and achieve zero order release. We have surprisingly found that in the case of tofacinib, by applying to the core a coating in a specific amount (measured in relation to the core tablet weight) that comprises a water soluble pH independent gelling control release polymer having a particular viscosity grade results in a zero-order release.

The monolithic tablet of the current invention provides similar drug dissolution release to an osmotic pump system by diffusion and erosion of Tofacitinib through the polymeric matrix. Moreover, the technology required for the manufacturing of a monolithic tablet is cheaper and as efficient as the one employed for obtaining osmotic pump systems. A further advantage is provided by using a simple coating which beside the water soluble pH independent gelling control release polymer having a particular viscosity grade does not require pore formers.

The monolithic tablet of the present invention comprises a core and a coating. The core comprising Tofacitinib and a water soluble pH independent gelling control release polymer; the coating is in an amount of 2.5% to 35.0% w/w in relation to the core tablet weight and comprises a water soluble pH independent gelling control release polymer. The pH independent gelling control release polymer in the core and in the coating of the current invention has a viscosity grade in a range from 50 to 150 cP in 2% solution in water at 20° C.

It was surprisingly found that the amount of the coating in combination with the specific viscosity of the water soluble pH independent gelling control release polymer in the core and in the coating strongly influences the dissolution profile of the monolithic tablet of the current invention.

The core of the controlled release pharmaceutical tablet of the invention comprises the whole dose of tofacitinib. The word tofacitinib is used herein to refer to tofacitinib free base as well as its pharmaceutically acceptable salts. A preferred salt to be used is the citrate salt.

Tofacitinib free base as well as its pharmaceutically acceptable salts, preferably tofacitinib citrate, is preferably used in an amount of 3% to 15%, more preferably 4% to 12%, most preferably 7% to 10% by weight based on the total inner tablet weight.

In the present invention tofacitinib is released from the formulation, in a controlled fashion so that after 2 hours less than 80% tofacitinib is released, at least 60% of tofacitinib is released after 4 hours and at least 80% of tofacitinib is released after 6 hours in USP III, 10 dpm, 250 ml, SIF pH 6.8, 37° C.

Alternatively USP III, 20 dpm, 250 ml, SIF pH 6.8, 37° C. dissolution method can be used. Using this method, after 2 hours less than 80% of tofacitinib is released, at least 60% of tofacitinib is released after 4 hours and at least 80% of tofacitinib is released after 6 hours.

In the present invention the core of the tablet contains at least one water soluble pH independent gelling control release polymer. The term water soluble pH independent gelling control release polymer means a control release polymer that forms a gel when in contact with water independently of the pH of the water. Such polymers are known in the art and include polyethylene oxide (for example (MW:900.000 g/mol: Polyox® 1105 WSR)), hydroxypropyl methylcellulose (for example Methocel® K100 Premium low viscosity (LV) grade), hydroxypropyl cellulose, polyvinyl alcohol (for example Parteck® SRP 80), guar gum, carrageenan and combinations thereof. A preferred pH independent gelling control release polymers are soluble polymers such a polyethylene oxide, hydroxypropyl methylcellulose, hydroxypropyl cellulose, polyvinyl alcohol and combinations thereof. More preferably a water soluble pH independent gelling control release polymer are polyethylene oxide and hydroxypropyl methyl cellulose, even more preferably a water soluble pH independent gelling control release polymer is hydroxypropyl methyl cellulose. The amount of the water soluble pH independent gelling control release polymer in the tablet core is preferably in an amount from 10% to 50%, more preferably from 10% to 40%, even more preferably from 15 to 35% by weight based on the total tablet core weight.

The water soluble pH independent gelling control release polymer in the core of the present invention has a viscosity grade in a range from 50 to 150 cP in 2% solution in water at 20° C., more preferably from 60 to 140 cP in 2% solution in water at 20° C., even more preferably from 70 to 130 cP in 2% solution in water at 20° C., most preferred from 80 to 120 cP in 2% solution in water at 20° C.: measured using a capillary viscosity method as described in USP monograph.

The tablet core may contain additional excipients such as fillers, glidants, lubricants, or buffering agents.

Fillers are excipients that are used to increase the bulk volume of a tablet. By combining a filler with the active pharmaceutical ingredient, the final product is given adequate weight and size to assist in production and handling.

The tablet core of the present invention contains preferably at least one filler.

Fillers are preferably used in an amount of from 40% to 85% more preferably 50% to 80% most preferably 50-70% by weight based on the total weight of the tablet core. Suitable examples of fillers to be used in accordance with the present invention include mannitol, sorbitol, microcrystalline cellulose, lactose, phosphates, hydroxypropyl cellulose, starch, pregelatinized starch, and combinations thereof.

In a preferred embodiment of the present invention, the fillers to be used are microcrystalline cellulose, lactose or mixtures thereof. In a further preferred embodiment of the present invention, the fillers to be used are microcrystalline cellulose and lactose.

In a preferred embodiment the proportion of the fillers when two are used is 50:50.

The tablet core may also contain glidants and/or lubricants.

Glidants enhance product flow by reducing interparticulate friction. A suitable example is colloidal silicon dioxide. Glidants are preferably used in a total amount of from 0.05% to 5%, more preferably 0.2% to 2%, most preferably 0.2% to 1.0% by weight based on the total weight of the tablet core.

Lubricants are generally used in order to reduce sliding friction. In particular, to decrease friction at the interface between a tablet's surface and the die wall during ejection, and reduce wear on punches and dies. Suitable lubricants to be used in accordance with the present invention include magnesium stearate, calcium stearate, stearic acid, glyceryl behenate, hydrogenated vegetable oil, and sodium stearyl fumarate. Lubricants are preferably used in a total amount of from 0.05% to 5%, more preferably 0.5% to 3%, most preferably 0.8% to 2.5% by weight based on the total weight of the tablet core. A preferred lubricant is magnesium stearate.

The tablet core may also contain one or more buffering agents. Buffering agents are generally used in order to maintain the pH constant. They may be acidic or basic agents. Suitable acidic buffering agents are tartaric acid, malic acid, maleic acid and citric acid. Suitable basic buffering agents are sodium carbonate, sodium acetate and potassium citrate.

In the present invention the tablet core is coated with a coating which retards the beginning of the drug release from the formulation. The coating comprises at least one water soluble pH independent gelling control release polymer.

The term water soluble pH independent gelling control release polymer means a control release polymer that forms a gel when in contact with water independently of the pH of the water. Such polymers are known in the art and include polyethylene oxide (for example (MW:900.000 g/mol: Polyox® 1105 WSR)), hydroxypropyl methylcellulose (for example Methocel® K100 Premium low viscosity (LV) grade), hydroxypropyl cellulose, polyvinyl alcohol (for example Parteck® SRP 80), guar gum, carrageenan and combinations thereof.

Suitable pH independent gelling control release polymers for the coating are polymers such a polyethylene oxide, hydroxypropyl methylcellulose, hydroxypropyl cellulose, polyvinyl alcohol and combinations thereof. Preferably, a water soluble pH independent gelling control release polymer in the coating is polyethylene oxide or hydroxypropyl methyl cellulose, even more preferably a water soluble pH independent gelling control release polymer in the coating is hydroxypropyl methyl cellulose.

The amount of the water soluble pH independent gelling control release polymer in the coating is preferably in an amount from 50% to 100% w/w, more preferably from 70% to 95% w/w based on the total tablet coating weight.

The coating of the current invention is in an amount of 2.5% to 35.0%, preferably 2.5% to 10, even more preferably 3 to 8% w/w in relation to the core tablet weight.

The water soluble pH independent gelling control release polymer in the coating of the present invention has a viscosity grade in a range from 50 to 150 cP in 2% solution in water at 20° C., more preferably from 60 to 140 cP in 2% solution in water at 20° C., even more preferably from 70 to 130 cP in 2% solution in water at 20° C., most preferred from 80 to 120 cP in 2% solution in water at 20° C.: measured using a capillary viscosity method as described in USP monograph.

The coating may be prepared using conventional methods well-known in the art. The coating is applied spraying a suspension of the coating components over the tablet. Such suspension is prepared by dispersing the coating components in a suitable solvent. Suitable solvents are purified water, ethanol, isopropyl alcohol, methylene chloride or mixtures thereof. Preferable suitable solvent is a mixture of ethanol:water in a ratio from 96:4 to 60:40, more preferable in a ratio from 90:10 to 70:30 and most preferable in a ratio of 80:20.

Optionally other excipients like plasticizer (e.g. polyethylene glycol, triacetin, hydroxypropyl cellulose and triethyl citrate), colourants (e.g. iron oxides, titanium dioxide) etc. are added obtaining a homogeneous suspension. The obtained suspension is sprayed over the tablets.

Further, the tablet of the invention shows a dissolution profile similar and it is bioequivalent to the commercial tofacitinib tablet XELJANZ XR®.

In a preferred embodiment the tablet comprises:
1. A core comprising based on total weight of the core weight:
   a. Tofacitinib or a pharmaceutically acceptable salt in an amount of from 3% to 15% w/w by weight;
   b. Hydroxypropyl methylcellulose in an amount of from 10% to 50% w/w by weight:
   c. One or more filler in an amount of from 40% to 85% w/w by weight;
   d. One or more glidant in an amount of from 0.2% to 1.0% w/w by weight;
   e. One or more lubricant in an amount of from 0.05% to 5% w/w by weight:
2. A coating in an amount of from 2.5% to 35% w/w in relation to the core tablet weight comprising hydroxypropyl methylcellulose;
and the hydroxypropyl methylcellulose in the core and in the coating has a viscosity grade in a range from 50 to 150 cP s in 2% solution in water at 20° C.

The tablet of the invention can be made using conventional methods and equipment well-known in the art: direct compression, wet granulation or dry granulation. In a preferred embodiment the tablet of the invention is prepared by direct compression.

Alternatively, the coating comprising a water soluble pH independent gelling control release polymer of the invention as described in all embodiments herein above can be in an amount of from 2.5% to 15% or from 2.5% to 10, even more preferably 3 to 8% w/w in relation to the core tablet weight.

Alternatively, the coating comprising a water soluble pH independent gelling control release polymer of the invention as described in all embodiments herein above can be 7, 4%, 5%, 7%, 10% in relation to the core tablet weight.

The tablet composition in accordance with the present invention is bioequivalent in vitro and in vivo to the commercially available tofacitinib citrate tablets.

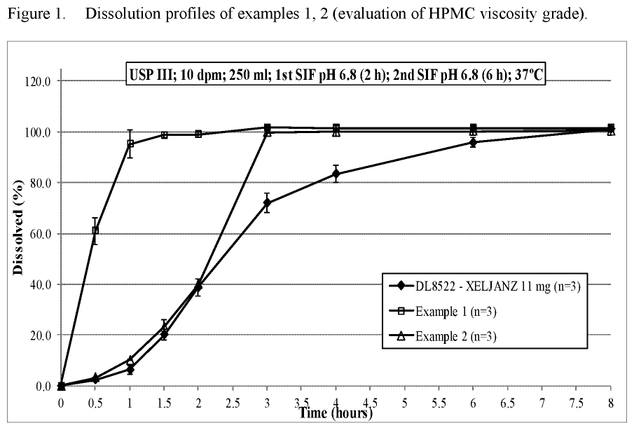

FIG. 1. Dissolution profiles of examples 1, 2 (evaluation of HPMC viscosity grade).

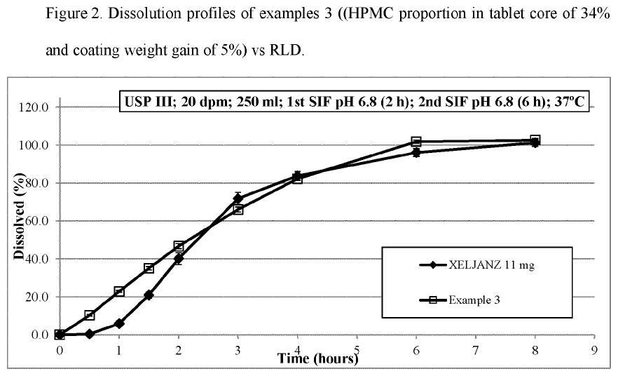

FIG. 2. Dissolution profiles of examples 3 (HPMC proportion in tablet core of 34% and coating weight gain of 5%).

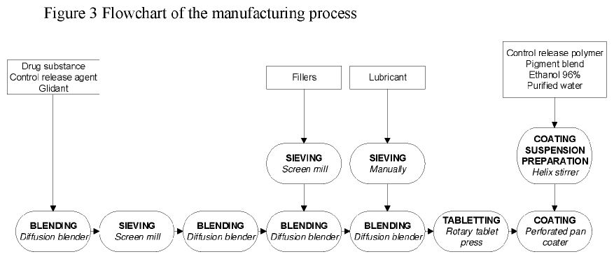

FIG. 3. Flowchart of the manufacturing process.

The present invention is illustrated by the following Examples. In table 1 the pharmaceutical composition of examples 1 and 2 are shown. In table 2 the pharmaceutical composition of example 3 is shown.

### Examples 1 & 2, Evaluation of HPMC Viscosity Grade: Prolonged Release Formulation Containing 15% w/w of Methocel K100 LV (HPMC 100 cP in 2% Solution at 20° C.) in the Tablet Core Coated with Methocel K3 (HPMC 3 cP in 2% Solution at 20° C.) or with Methocel K100 LV (HPMC 100 cP in 2% Solution at 20° C.) in Examples 1 and 2 Respectively. Coating Weight Increase of 15% w/w Relative to Tablet Core

One batch of 700 g of tablets cores was manufactured at lab scale. To do that 62.2 grams of tofacitinib citrate, 105.0 grams of Methocel K100 LV CR and 3.5 grams of Aerosil 200VV Pharma are weighed and de-agglomerated through a sieve of 1.0 mm mesh size. The components are mixed in a diffusion blender at 72 rpm for 10 minutes obtaining a homogenous blend (1). 261.2 grams of microcrystalline cellulose and 261.2 grams of lactose monohydrate are weighed, de-agglomerated through a sieve of 1.0 mm mesh size and then added together with the previous blend (1): the components are mixed in a diffusion blender at 20 rpm for 10 minutes, obtaining a homogenous blend (2). 7 grams of Magnesium stearate are weighed and de-agglomerated using a sieve of 0.5 mm mesh size and added to the previous blend (2); the components are mixed in a diffusion blender at 20 rpm for 3 minutes, resulting in a homogenous blend (3). This blend (3) is then compressed in a rotary tabletting machine, obtaining tablets with appropriate hardness (≈150 N).

To prepare example 1, 230 g of tablet cores are added to the coater pan. Coating suspension is prepared in excess (150%) for the coating of the tablets. 51.8 grams of Methocel K3 LV are weighed and added into 983.3 grams of purified water, mixed with a helix stirrer during at least 45 min. Then the suspension is sprayed over the tablets previously heated in the coating pan, until the tablets achieved a 15% w/w weight increase.

To prepare example 2, 200 g of tablet cores are added to the coater pan. Coating suspension is prepared in excess (200%) for the coating of the tablets. 60.0 grams of Methocel K100 LV CR are weighed and added into 2280 grams of purified water, mixed with a helix stirrer during at least 45 min. Then the suspension is sprayed over the tablets previously heated in the coating pan, until the tablets achieved a 15% w/w weight increase.

Dissolution profiles of examples 1, 2 and of XELJANZ XR® can be seen in FIG. 1.

TABLE 1

| | Example 1 | Example 2 |
|---|---|---|
| | mg weight/ | mg weight/ |
| | tablet | tablet |
| TOFACITINIB citrate | 17.76 | 17.76 |
| Hydroxypropyl methylcellulose (Hypro- | 30.00 | 30.00 |
| mellose 2208, 100 cP, 2% solution at 20° C.) | | |
| Colloidal silicon dioxide | 1.00 | 1.00 |
| Mycrocrystalline cellulose | 74.62 | 74.62 |
| Lactose monohydrate | 74.62 | 74.62 |
| Magnesium stearate | 2.00 | 2.00 |
| Total tablet core weight (mg) | 200.00 | 200.00 |
| Hydroxypropyl methylcellulose (Hypro- | 30.00 | — |
| mellose 2208, 3 cP, 2% solution at 20° C.) | | |
| Hydroxypropyl methylcellulose (Hypro- | — | 30.00 |
| mellose 2208, 100 cP, 2% solution at 20° C.) | | |
| Coated tablet | 230.00 | 230.00 |

The above formulations were made according to the process depicted in FIG. 3.

### Example 3: Prolonged Release Formulation Containing 34% w/w of Methocel K100 LV (HPMC 100 cP in 2% Solution at 20° C.) in the Tablet Core Coated with Methocel K100 LV (HPMC 100 cP in 2% Solution at 20° C.) Up to Coating Weight Increase of 5% w/w Relative to Tablet Core

One batch of 8000 g of tablets cores was manufactured at lab scale. To do that 710.6 grams of tofacitinib citrate, 2720.0 grams of Methocel K100 LV CR and 40.0 grams of Aerosil 200VV Pharma are weighed The components are mixed in a diffusion blender at 20 rpm for 5 minutes and de-agglomerated through a sieve of 1.1 mm mesh size. Thereafter, the de-agglomerated material is mixed in a diffusion blender at 20 rpm for 5 minutes more obtaining a homogenous blend (1). 2184.7 grams of microcrystalline cellulose and 2184.7 grams of lactose monohydrate are weighed, de-agglomerated through a sieve of 1.1 mm mesh size and then added together with the previous blend (1); the components are mixed in a diffusion blender at 20 rpm for 10 minutes, obtaining a homogenous blend (2). 160.0 grams of Magnesium stearate are weighed and de-agglomerated using a sieve of 0.5 mm mesh size and added to the previous blend (2); the components are mixed in a diffusion blender at 20 rpm for 3 minutes, resulting in a homogenous blend (3). This blend (3) is then compressed in a rotary tabletting machine, obtaining tablets with appropriate hardness (≈120 N).

3000 g of tablet cores produced are added to the coater pan. Coating suspension is prepared in excess (30%) for the coating of the tablets. 156.0 grams of Methocel K100 LV CR and 39.0 g of pigment blend (containing colorants and plasticizer) are weighed and added into 2964.0 grams of ethanol and mixed with a helix stirrer during at least 5 min. Then 741.0 g of purified water is added over the previous suspension and mixed with a helix stirrer during at least 60 minutes more. Then the suspension is sprayed over the tablets previously heated in the coating pan. Once the tablets achieved a 5% weight increase, the process is finished.

Example 3
mg weight/tablet

Tablet core components

| | |
|---|---|
| TOFACITINIB citrate | 17.76 |
| Hydroxypropyl methylcellulose | 68.00 |
| (Hypromellose 2208, 100 cP, 2% solution at 20° C.) | |
| Colloidal silicon dioxide | 1.00 |
| Mycrocrystalline cellulose | 54.62 |
| Lactose monohydrate | 54.62 |
| Magnesium stearate | 4.00 |
| Total tablet core weight (mg) | 200.00 |

Coating components

| | |
|---|---|
| Hydroxypropyl methylcellulose | 8.00 |
| (Hypromellose 2208, 100 cP, 2% solution at 20° C.) | |
| Pigment blend | 2.00 |
| Coated tablet | 210.00 |

Dissolution profiles of example 3 and XELJANZ XR can be seen in FIG. 2.

1. A controlled release pharmaceutical tablet comprising:
a) a core comprising tofacitinib or a pharmaceutically acceptable salt thereof and a water soluble pH independent gelling control release polymer; and
b) a coating in an amount of 2.5% to 35.0% w/w in relation to the core tablet weight comprising a water soluble pH independent gelling control release polymer;
wherein the pH independent gelling control release polymer in the core and in the coating has a viscosity grade in a range from 50 to 150 cP in 2% solution in water at 20° C.

2. The tablet according to claim 1 wherein tofacitinib is present in an amount of from 3% to 15% by weight based on the total tablet core weight.

3. The tablet according to claim 1 wherein said water soluble pH independent gelling control release polymer in said core is in an amount from 10% to 50% by weight to the total tablet core weight.

4. The tablet according to claim 1 wherein said water soluble pH independent gelling control release polymer in said core is selected from the group consisting of polyethylene oxide, hydroxypropyl methylcellulose, hydroxypropyl cellulose, polyvinyl alcohol, and combinations thereof.

5. The tablet according to claim 4, wherein said water soluble pH independent gelling control release polymer in said core is hydroxypropyl methylcellulose.

6. The tablet according to claim 1 wherein said water soluble pH independent gelling control release polymer in said coating is selected from the group consisting of polyethylene oxide, hydroxypropyl methylcellulose, hydroxypropyl cellulose, polyvinyl alcohol, and combinations thereof.

7. The tablet according to claim 6, wherein said water soluble pH independent gelling control release polymer in said coating is hydroxypropyl methylcellulose.

8. The tablet according to claim 1 wherein said core further comprises one or more excipients selected from the group consisting of filler, glidant, lubricant and buffering agent.

9. The tablet according to claim 1; wherein said core comprises based on total weight of the core weight:
a) tofacitinib or a pharmaceutically acceptable salt in an amount of from 3% to 15% w/w by weight;
b) hydroxypropyl methylcellulose in an amount of from 10% to 50% w/w by weight;
c) one or more filler in an amount of from 40% to 85% w/w by weight;
d) one or more glidant in an amount of from 0.2% to 1.0% w/w by weight;
e) one or more lubricant in an amount of from 0.05% to 5% w/w by weight;
and wherein said coating is in an amount of from 2.5% to 35% w/w in relation to the core tablet weight and comprises hydroxypropyl methylcellulose and the hydroxypropyl methylcellulose in the core and in the coating has a viscosity grade in a range from 50 to 150 cP in 2% solution in water at 20° C.

10. The tablet according to claim 9 wherein the hydroxypropyl methylcellulose in the core and in the coating has a viscosity grade in a range from 80 to 120 cP in 2% solution in water at 20° C.

11. The tablet according to claim 9 wherein said filler is selected from the group comprising mannitol, sorbitol, microcrystalline cellulose, lactose, phosphates, hydroxypropyl cellulose, starch and combinations thereof.

12. The tablet according to claim 11 wherein said filler is a combination of microcrystalline cellulose and lactose.

13. The tablet according to claim 1 wherein tofacitinib is in the form of tofacitinib citrate.
